(19) United States
(12) Plant Patent Application Publication
(10) Pub. No.: US 2015/0351301 P1
(43) Pub. Date: Dec. 3, 2015

(54) Dianthus plant named `KLEDG13160'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/120,503

(22) Filed: May 27, 2014

(51) Int. Cl. A01H 5/00

(52) U.S. Cl. PLT/283

(57) ABSTRACT

A new Dianthus plant particularly distinguished by having single, red bicolored flowers, a compact growth habit, and strong branching, is disclosed.

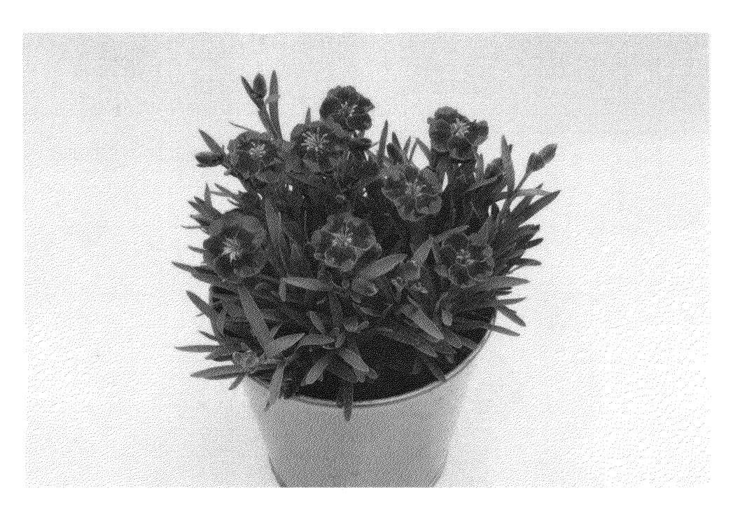

### GENUS AND SPECIES

Dianthus sp.

### VARIETY DENOMINATION

‘KLEDG13160’

### BACKGROUND OF THE NEW PLANT

The present invention comprises a new and distinct variety of Dianthus, botanically known as Dianthus sp. and hereinafter referred to by the variety name ‘KLEDG13160’. ‘KLEDG13160’ is a selection that results from a cross pollination of the proprietary female dianthus line ‘DSS 2008 0052’ (unpatented) and the male dianthus line ‘KLEDG13146’ (U.S. Plant patent Applied For).

In June of 2009 the proprietary female dianthus line ‘DSS 2008 0052’ and the male dianthus line ‘KLEDG13146’ were cross pollinated in Stuttgart, Germany and seeds were obtained. The seeds were sown and plants were grown for evaluation. A plant line was selected in June 2010 in Stuttgart, Germany, and named ‘KLEDG13160’. In June 2010, ‘KLEDG13160’ was first vegetatively propagated by vegetative cuttings. ‘KLEDG13160’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. Single, red bicolored flowers;

2. Compact growth habit; and

3. Strong branching.

### CROSS REFERENCE TO RELATED APPLICATIONS

Plant Breeder's Rights for ‘KLEDG13160’ were applied for in in Europe with the Community Plant Variety Office on Jul. 1, 2013, application number 2013/1809. ‘KLEDG13160’ has not been made publicly available or sold more than one year prior to the filing date of this application.

### DESCRIPTION OF THE PHOTOGRAPH

This new Dianthus plant is illustrated by the accompanying photograph which shows an overall view of the plant. The colors shown are as true as can be reasonably obtained by conventional photographic procedures. The plant is about 4 to 5 months-old grown indoors in Stuttgart, Germany in April 2011.

### DESCRIPTION OF THE NEW VARIETY

The following detailed descriptions set forth the distinctive characteristics of ‘KLEDG13160’. The data which define these characteristics were collected from asexual reproductions carried out in Stuttgart, Germany. Data was collected on four month-old plants grown in a greenhouse in Stuttgart, Germany in March 2014. Color references are to The R.H.S. Colour Chart of The Royal Horticultural Society of London (R.H.S.) 5^{th} edition (2007).
Classification:
   Family.—Caryophyllaceae.
   Species.—Dianthus sp.
   Common name.—Garden carnation.
   Variety.—‘KLEDG13160’.
Parentage:
   Female.—The proprietary dianthus ‘DSS 2008 0052’.
   Male.—The dianthus ‘KLEDG13146’ (U.S. Plant patent Applied For).
Propagation:
   Type.—Vegetative cuttings.
   Time to produce a rooted young plant.—20 days.
   Root description.—White, well-branched, fine.
   Rooting habit.—Lanceolate.
Plant description:
   Plant form.—Potted plant, upright, bushy, compact.
   Plant height.—9.0 cm.
   Plant diameter or spread.—15.0 cm.
Lateral branches:
   Length.—5.0 cm.
   Diameter.—2.0 cm.
   Internode length.—0.5 cm.
   Node length.—0.3 cm.
   Node diameter.—0.3 cm.
   Texture.—Smooth.
   Color.—RHS N138B.
Foliage description:
   Arrangement.—Opposite.
   Length.—4.0 cm.
   Width.—0.4 cm.
   Shape.—Lanceolate.
   Apex.—Acute.
   Base.—Obtuse.
   Margin.—Entire.
   Texture.—Upper surface: Smooth.
   Venation pattern.—Pinnate.
   Color.—Immature and mature leaves: Upper surface: RHS N138A Lower surface: RHS N138B Venation: RHS N138D.
Flower description:
   Flower type and habit.—Single, round.
   Fragrance.—Present.
   Natural flowering season.—Spring to Fall.
   Flower longevity.—Medium, 4 to 6 days.
   Flower buds.—Length: 1.2 cm Diameter: 0.7 cm Shape: Ovate Color: Mixed, RHS 183C and RHS 137C.
   Corolla.—Quantity of petals/arrangement: 5, radial Corolla height: 1.0 cm Petal length: 3.0 cm Petal width: 1.8 cm Petal shape: V-shaped Petal apex: Obtuse Petal base: Cuspidate Petal margin: Slightly serrate Petal texture: Upper and lower surfaces: Smooth Petal color: Immature: Upper surface: RHS 59A to RHS 59C Lower surface: RHS 64A Mature: Upper surface: RHS 59A to RHS 59C Lower surface: RHS 64C to RHS 64D Calyx: Quantity of sepals/arrangement: 5, single Calyx height: 1.3 cm Calyx diameter, at the apex: 0.6 cm Sepal texture: Upper and lower surfaces: Smooth Sepal color: Upper surface: RHS 187B and RHS 137C Lower surface: RHS 137B.
Peduncles:
   Length.—7.0 cm.
   Diameter.—0.2 cm.
   Strength.—Moderate to strong.
   Texture.—Smooth.
   Color.—RHS N138D.
Reproductive organs:
   Stamens.—Quantity: Moderate to sparse Filament length: 1.0 cm Filament color: RHS 59A Anther length: 0.2 cm Anther diameter: 0.08 cm Anther color: RHS 84D Pollen amount: Moderate to sparse.
   Pistils.—Quantity: 2 Pistil length: 1.6 cm Style length: 0.1 cm Style color: RHS 73D Stigma shape: Filamentous Stigma color: RHS 59A Ovary size: 0.4 cm Ovary shape: Ovate Ovary color: RHS 139D Fruits/seeds: None observed.
Disease and insect/pest resistance: No special observations were made

### COMPARISON WITH PARENTAL LINES AND KNOWN VARIETY

‘KLEDG13160’ is a distinct variety of dianthus. ‘KLEDG13160’ is distinguished from its parents as described in Table 1:

TABLE 1
Comparison with Parental Lines

| | Variety | Female parent | Male Parent |
|---|---|---|---|
| Trait | ‘KLEDG13160’ | ‘DSS 2008 0052’ | ‘KLEDG13146’ |
| Flower type | Single | Double | Double |
| Growth | Compact | Medium to small | Medium |
| Branching | Strong | Less | Very strong |

‘KLEDG13160’ is most similar to the commercial dianthus plant named ‘KLEDG11143’ (U.S. Plant Pat. No. 23,150), commercially known as Everlast ‘Burgundy Blush’. Differences between the two varieties are described in Table 2:

TABLE 2
Comparison with Similar Variety

| | Variety | Commercial line |
|---|---|---|
| Trait | ‘KLEDG13160’ | ‘KLEDG11143’ |
| Flower type | Single | Double |
| Earliness | Medium | Medium to late |
| Branching | Strong | Medium |

We claim:

1. A new and distinct variety of Dianthus plant named ‘KLEDG13160’ as shown and described herein.
